(19) United States
(12) Patent Application Publication
(10) Pub. No.: US 2026/0237487 A1
(43) Pub. Date: Aug. 13, 2026

(54) TRAINING DATA FOR 3D IMAGE CLEANING ML MODEL

(71) Applicant: KONINKLIJKE PHILIPS N.V., EINDHOVEN (NL)

(72) Inventors: Sergey Kastryulin, Moscow (RU); Christian Wuelker, Hamburg (DE); Nils Thorben Gessert, Hamburg (DE); Nicola Pezzotti, Eindhoven (NL); Alexander Selivanov, Unterschleißheim (DE); Tim Nielsen, Hamburg (DE)

(21) Appl. No.: 19/150,944

(22) PCT Filed: Jan. 24, 2024
(86) PCT No.: PCT/EP2024/051580

§ 371 Date: Jul. 25, 2025

(30) Foreign Application Priority Data
Feb. 1, 2023 (RU) 2023102210

(51) Int. Cl. G16H 30/40; G06T 3/40; G06T 5/60; G06T 7/10

(52) U.S. Cl. CPC G16H 30/40; G06T 3/40; G06T 5/60; G06T 7/10; G06T 2207/20084; G06T 2207/20132

(57) ABSTRACT

Disclosed herein is a method for creating training data for a machine learning model. The machine learning model is configured for improving quality of an acquired 3D medical image. The method comprises: selecting videos having desired values of one or more image parameters; pre-processing the videos of the dataset. The pre-processing comprises down sampling the videos by a predefined reduction factor, cropping the down sampled videos in space and/or time to obtain video patches, herein referred to as clean video patches, altering the clean video patches for reducing a quality of the clean video patches, creating a training dataset comprising the altered video patches as inputs and associated clean video patches as learning targets.

### FIELD OF THE INVENTION

The invention relates to magnetic resonance imaging, in particular to creating and using data for training a machine learning model for cleaning 3D medical image data.

### BACKGROUND OF THE INVENTION

Choosing the training data for healthcare artificial intelligence (AI) models is a basic and important part of the whole workflow. However, training of AI models on magnetic resonance (MR) scans may be problematic as the provided training data may not be suitable for obtaining improved models.

### SUMMARY OF THE INVENTION

The invention provides for a medical system, a computer program, data structure and a method in the independent claims. Embodiments are given in the dependent claims.

Training of AI models on MR scans may be problematic as the training data is usually of small quantity, and the quality and representation of the data may be poor. This is because, the acquisition of high-quality MR data may be challenging and time consuming and sometimes may be impossible. Embodiments may use natural high-quality videos as a training data source for training three-dimensional (3D) AI image quality improvement models for 3D MR scans. The advantage of this approach may be in the diversity of the data. Videos may be of high-quality with no compression, noise, or interpolation artefacts, which can serve as good reference data. Special pre-processing and data augmentation techniques may be provided for enabling the use of such data for 3D image quality improvement. This approach may be advantageous for denoising, anti-ringing and super resolution tasks for MR scans in image quality (IQ) Boost 3D and it could be extended to other modalities and tasks.

In one aspect the invention provides for a medical system that comprises a memory storing machine executable instructions, and a computational system, wherein execution of the machine executable instructions causes the computational system to: receive videos from one or more sources, select, from the received videos, videos having desired values of one or more image parameters; create a dataset (initial dataset) comprising the selected videos, and pre-process the videos of the dataset. The pre-processing comprises: down sampling the videos by a predefined reduction factor; cropping the down sampled videos in space and/or time to obtain video patches, herein referred to as clean video patches; altering the clean video patches for reducing a quality of the clean video patches. A training dataset may be created. The training dataset comprises the altered video patches as inputs and associated clean video patches as learning targets.

The videos may, for example, be down sampled along the spatial dimensions. Alternatively, the videos may be down sampled along the time dimension. This may particularly be advantageous in case of slow-motion videos.

The machine learning model may be configured to enhance a quality of acquired 3D image data such as 3D MR image data. The quality of the image data may include the level of noise in the image data, the resolution of the image data, the number of artifacts in the image data etc. The enhancing may include denoising, anti-ringing, super resolution, motion artifact reduction, ghosting artifact removal or undersampling artifact removal. The machine learning model may, for example, be a fully convolutional neural network with 3D kernels, ResNet, DenseNet, EfficientNet, or Vision Transformer with adjusted kernels for 3D processing.

The videos may be received from one or more sources. The videos may be treated as three dimensional volumes where the third dimension is time. The sources may comprise web sources such as social media websites or public databases e.g., with appropriate license policy. The received videos may represent non-medical fields. For example, the received videos do not comprise medical images such as magnetic resonance (MR) images. For example, the received videos may be Natural high-quality Videos (NV). Training on natural videos may increase generalizability of the machine learning model. The machine learning model may learn more valuable features from non-MRI content. Using the received videos, the machine learning model may not learn to overfit to any anatomy structure; hence this may increase the confidence that the anatomical structure will remain untouched during inference. Moreover, the received videos such as NV may be available in large quantities and there may be many of them in high-resolution that can serve as good references for the machine learning model. In addition, the received videos may be easy to get for research purposes as there are less risks for data privacy issues comparing to medical domain data. Since the machine learning model can be trained with a lot of high-quality, non-medical examples, it can perform well on other domain data during inference. In addition, using videos from the non-medical field may be advantageous for the following reasons. Acquiring high-quality medical images (e.g., 3D MR images) for training AI models may be very costly and sometimes impossible. For example, acquiring a completely noise-free medical image may be impossible. The noise can be reduced to some degree by using multiple medical image acquisitions and averaging. However, for in-vivo scans, this may lead to motion of the subject and therefore reduced image quality. The present videos may overcome these issues related to 3D MR images.

The present subject matter may further improve the training of the machine learning model based on said received videos by selecting relevant videos, down sampling the selected videos and creating video patches from the down sampled videos. The down sampling step followed by the cropping step may form the pre-processing (or processing) steps.

The down sampling of the video may be performed along the spatial dimensions of the video or along the time dimension of the video. The down sampling of the video may, for example, comprise down sampling each frame of said video to a smaller size. For example, the frame may have an initial size X0×Y0 and may be down sampled to the size of X1×Y1, where X1<X0 and Y1<Y0. This may save memory space required to store each frame of the video while providing 3D image data which has a quality better than or at least similar to the quality of acquired medical image data. Another purpose of the downsampling may be to match the scale of the image content to MRI-like image content. For example, a patch of size 50×50, cropped from a 4 k resolution image may contain less structure and content than a 50×50 crop from full-hd resolution image. Thus, the downsampling factor can be chosen such that the resulting image content and scale are similar to MRI image content.

Each down sampled video may be cropped. This may result in a set of video patches, referred to as set of clean video patches. Video patches may be randomly cropped from respective down sampled videos. The video patch is a video. The video patch may be entirely contained within its source video. The video patches obtained from the same video may or may not overlap. In one example, the overlap between video patches obtained from the same video may not exceed 20%. This may enable to cover the whole 3D space occupied by the video and thus covering all features in the video. For example, three different kinds of video patches may be obtained from each down sampled video of the dataset. A spatial video patch may be obtained by cropping in space a given video, such that the spatial video patch has the same temporal duration as the given video, but cropped to e.g., 30%, of spatial dimensions. A temporal video patch may be obtained by cropping in time a given video, such that temporal video patch has the same spatial size as the given video, but clipped to e.g., 30%, of temporal duration. A spatial-temporal video patch may be obtained by cropping in time and space a given video, such that the cropping is performed to e.g., 30%, along all three dimensions of the given video. The video patches may enable to augment the training data and capture the local quality features of videos. This may enable to improve medical image cleaning as the patch-based training may lead to more generalizable AI models.

Hence, the cropping of the down sampled videos may provide the set of clean video patches. Due to their improved quality, these clean video patches may advantageously be used as targets or labels for training the machine learning model. The inputs of the training of the machine learning models may be obtained by altering the set of clean video patches. The pre-processing may further comprise the altering step. The alteration of the set of clean video patches may result in the set of altered video patches in association with the respective clean video patches. The present subject matter may alter the video patch based on the purpose of the machine learning model. For example, if the machine learning model is for denoising the medical images, the alteration may be performed by adding noise to the video patch. The training dataset may be created using the set of altered video patches as inputs and the associated set of clean video patches as targets.

The present subject matter may be advantageous as it may enable image quality enhancement e.g., by denoising the images more accurately, using the machine learning model after being trained using the present training dataset. That is, when used, the present training dataset may enable to improve the cleaning of 3D medical images. The present subject matter may provide reliable training datasets in desired amounts without relying on medical images whose acquisition may take time and cannot provide very high-quality medical images which may be needed for training. The cleaning of the 3D medical image may, for example, comprise denoising, super-resolution, sharpening, motion artifact reduction, ghosting artifact removal or undersampling artifact removal.

The present subject matter may prevent using a 2D AI image quality improvement model and apply it slice-wise to the 3D MRI scan. Indeed, due to the very small slice thickness of 3D MRI scans, the application of a 2D model slice-by-slice, may lead to inconsistencies between slices that reduce quality and degrade the scan. Therefore, an IQ improvement model for 3D MRI acquisitions may require a 3D model trained on 3D data as provided by the present subject matter.

According to one embodiment, the execution of the machine executable instructions causes the computational system to permute the dimensions in selected video patches of the set of clean video patches before the alteration of the set of clean video patches. For example, before altering the set of clean video patches, a subset of one or more videos patches may be randomly selected from the set of clean video patches. The dimensions of each selected video patch may be permuted. The resulting set of clean video patches includes permuted video patches and non-permuted video patched. The resulting set of clean video patches may be altered. This may result in a set of altered video patches associated with the set of clean video patches respectively. This embodiment may enable to mix dimensions so that the machine learning model does not get biased toward the time dimension. This may also enable to have all data equally processed in different dimensions.

According to one embodiment, the execution of the machine executable instructions causes the computational system to flip selected video patches of the set of clean video patches before the alteration of the set of clean video patches. For example, before altering the set of clean video patches, a subset of one or more videos patches may be randomly selected from the set of clean video patches. Each selected video may be flipped. The resulting set of clean video patches includes flipped video patches and non-flipped video patched. The resulting set of clean video patches may be altered.

By randomly permuting and/or flipping, the overfitting to properties of the temporal dimension by the machine learning model may be prevented. The permutation and/or flipping may make the video more similar to 3D MR data properties where each image dimension has similar properties.

In one example, the execution of the machine executable instructions causes the computational system to both permute and flip a subset of selected video patches of the set of clean video patches before the alteration of the resulting set of clean video patches. Alternatively, the execution of the machine executable instructions causes the computational system to permute a first subset of selected video patches of the set of clean video patches and to flip a different second subset of selected video patches of the set of clean video patches before the alteration of the resulting set of clean video patches. These examples may further improve the data representation of the training dataset.

According to one embodiment, each video of the initial dataset may have at least one of the following characteristics: a resolution higher than a minimum resolution, missing artifacts from compression or interpolation, an amount of noise smaller than a maximum amount, and the variation of the gradients is larger than a minimum amount. For example, this embodiment may define the following image parameters: resolution, number of artifacts, amount of noise, and gradients in each direction. The target (or desired) value of the resolution parameter may be a resolution higher than the minimum resolution. The target value of the number of artifacts may be zero. The target value of the amount of noise may be an amount of noise smaller than a maximum amount. The variation of the gradients for each direction is larger than a minimum amount. The last point means that the videos should contain some sort of motion and do not comprise of static images over time. This initial selection of videos may enable to obtain a reliable training dataset for an accurate training of machine learning models. This may enable to improve medical image cleaning using such trained model. The cleaning may, for example, comprise denoising, super-resolution, sharpening, motion artifact reduction, ghosting artifact removal or undersampling artifact removal.

According to one embodiment, the execution of the machine executable instructions causes the computational system to alter the video patch by adding a random Gaussian noise to the video patch or lowering a resolution of the video patch. The lowering of the resolution may be performed, for example, by applying a crop in k-space of the video patch with keeping specific percentage of information of k-space. The k-space cropping of the video patch may be performed to obtain a correspondence of the videos with the properties of the k-space of acquired MR data. For example, the k-space cropping may enable a mapping between data spacing in k-space and the cropped video patch. The application of the k-space crop may result in a low-resolution video and ringing artifacts in the video that serves as an input to the machine learning model. The resolution reduction may also be caused by downsampling and the interpolation (e.g., using bspline).

The alteration is performed based on the purpose of the machine learning model. As an example, if the machine learning model is a 3D AI denoiser, the machine learning model may be trained by adding random Gaussian noise to the pre-processed videos which are then used as input to the machine learning model. The learning target is the clean pre-processed video without noise. In this case, the training may be performed with standard gradient descent methods. In another example, if the machine learning model is a 3D AI anti-ringing and super-resolution model, this model may be trained by applying a k-space crop to the pre-processed video, resulting in a low-resolution video with ringing artefacts that serves as an input to the model. The learning target is the clean, pre-processed video.

According to one embodiment, the execution of the machine executable instructions causes the computational system to randomly perform the crop such that each video patch has a same size that is smaller than a maximum size. This embodiment may deal with huge volume sizes in a systematic manner using a small but fixed patch size. The random cropping may prevent the machine learning model from overfitting to specific features.

According to one embodiment, the execution of the machine executable instructions causes the computational system to apply an interpolator for down-sampling the videos. This may enable to preserve good quality of the video after down-sampling. The interpolator may, for example, be a high-quality interpolator such as Lanczos or other high-quality interpolators that can be used for down-sampling the received videos.

According to one embodiment, the execution of the machine executable instructions causes the computational system to: augment the dataset by resampling each video of the initial dataset to obtain different versions of the video, wherein each version of the video has a different size. The obtained videos are added to the initial dataset before the videos in dataset are pre-processed. This may further increase the size and diversity of the training dataset and thus enable accurate medical image cleaning. The cleaning may, for example, comprise denoising, super-resolution, sharpening, motion artifact reduction, ghosting artifact removal or undersampling artifact removal.

According to one embodiment, the reduction factor is defined in accordance with a resolution of the medical images used by the machine learning model. For example, the videos may be down sampled to a size which is the size of 3D medical images used by the machine learning model during inference. The 3D medical images may, for example, be MRI images or computed tomography (CT) images. For example, the reduction factor is 2, 3 or 4. These values may, for example, be user defined e.g., empirically defined values that provide the most optimal down sampling result. Having predefined fixed reduction factors may enable systematic down sampling of the videos and save resources that may be required for computing them dynamically.

According to one embodiment, the execution of the machine executable instructions causes the computational system to train the machine learning model using the training dataset. In one example, the trained machine learning model may be inferred in order to improve quality of input 3D medical images. The input 3D medical images may be MR images, Ultrasound images or CT images.

According to one embodiment, the medical system further comprises a magnetic resonance imaging system, wherein the memory further contains pulse sequence commands configured to control the magnetic resonance imaging system to acquire k-space data according to the magnetic resonance imaging protocol, wherein the execution of the machine executable instructions further causes the computational system to: acquire the k-space data by controlling the magnetic resonance imaging system; and reconstruct 3D magnetic resonance images from the k-space data; use the trained model to enhance quality of the 3D magnetic resonance images. The use of the trained machine learning model comprises inference of the machine learning model with the reconstructed 3D MR images. This may enable a reliable and accurate MR imaging system.

Natural image (e.g., of the received videos) may not have complex part in it, while MR scans do. To overcome this during inference, real and imaginary parts of the MR scan may be treated and processed by models as individual images and both outputs are combined to get the result by the end.

According to one embodiment, the received videos are videos of non-medical fields. This may be advantageous compared to medical images for the following reasons. The received videos may provide representative, high quality, quantity enough data for training. However, in MRI medical domain, the existing MRI datasets may lack of quality, quantity and they may usually be biased. There may be no MRI data collected in high resolution at all, as there are no such scanners that can collect data in 4K resolution for example. MR scans are affected by noise and Gibb's ringing artifacts, they have a low resolution and low perceived sharpness, resulting in reduced image quality. It may also be not secure to train on MRI data, as the model can learn anatomy structures (such as brain/knee structure) and perform on them during inference, bringing the unexpected core changes. By using videos such as Natural Videos (NV), these problems can be overcome. The resolution of data is higher, hence more information contained in a single piece of data. The amount of data can be 10-100 times more than with MRI data. NV may contain every possible structure and geometry, not only MRI specific. Hence the representativeness of data is much higher.

In another aspect the invention relates to a computer program comprising machine executable instructions for execution by a computational system; wherein execution of the machine executable instructions causes the computational system to: receive videos from one or more sources; select, from the received videos, videos having desired values of one or more image parameters; create a dataset comprising the selected videos; pre-process the videos of the dataset, wherein the pre-processing comprises: down sampling the videos by a predefined reduction factor; cropping the down sampled videos in space and/or time to obtain video patches, herein referred to as clean video patches; and altering the clean video patches for reducing a quality of the clean video patches, and create a training dataset comprising the altered video patches as inputs and associated clean video patches as learning targets. The videos may, for example, be down sampled along the spatial dimensions of the videos or along the time dimension of the videos.

In another aspect the invention relates to a method for creating training data for a machine learning model, the machine learning model being configured for improving quality of an acquired 3D medical image. The method comprises:
receiving videos from one or more sources;
selecting, from the received videos, videos having desired values of one or more image parameters;
creating a dataset comprising the selected videos;
pre-processing the videos of the dataset, the pre-processing comprising
down sampling the videos by a predefined reduction factor; cropping the down sampled videos in space and/or time to obtain video patches, herein referred to as clean video patches;
altering the clean video patches for reducing a quality of the clean video patches;
creating a training dataset comprising the altered video patches as inputs and associated clean video patches as learning targets. The videos may, for example, be down sampled along the spatial dimensions of the videos or along the time dimension of the videos.

In another aspect the invention relates to a computer implemented data structure for training a machine learning model for cleaning 3D medical images, the data structure comprising altered video patches and associated clean video patches, wherein the altered video patch has an alteration obtained in accordance with a cleaning purpose of the machine learning model. The cleaning of the 3D medical image may, for example, comprise denoising, super-resolution, sharpening, motion artifact reduction, ghosting artifact removal or undersampling artifact removal from the 3D medical image.

It is understood that one or more of the aforementioned embodiments of the invention may be combined as long as the combined embodiments are not mutually exclusive.

As will be appreciated by one skilled in the art, aspects of the present invention may be embodied as an apparatus, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer executable code embodied thereon.

Any combination of one or more computer readable medium(s) may be utilized. The computer readable medium may be a computer readable signal medium or a computer readable storage medium. A ‘computer-readable storage medium’ as used herein encompasses any tangible storage medium which may store instructions which are executable by a processor or computational system of a computing device. The computer-readable storage medium may be referred to as a computer-readable non-transitory storage medium. The computer-readable storage medium may also be referred to as a tangible computer readable medium. In some embodiments, a computer-readable storage medium may also be able to store data which is able to be accessed by the computational system of the computing device. Examples of computer-readable storage media include, but are not limited to: a floppy disk, a magnetic hard disk drive, a solid-state hard disk, flash memory, a USB thumb drive, Random Access Memory (RAM), Read Only Memory (ROM), an optical disk, a magneto-optical disk, and the register file of the computational system. Examples of optical disks include Compact Disks (CD) and Digital Versatile Disks (DVD), for example CD-ROM, CD-RW, CD-R, DVD-ROM, DVD-RW, or DVD-R disks. The term computer readable-storage medium also refers to various types of recording media capable of being accessed by the computer device via a network or communication link. For example, data may be retrieved over a modem, over the internet, or over a local area network. Computer executable code embodied on a computer readable medium may be transmitted using any appropriate medium, including but not limited to wireless, wire line, optical fiber cable, RF, etc., or any suitable combination of the foregoing.

A computer readable signal medium may include a propagated data signal with computer executable code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

‘Computer memory’ or ‘memory’ is an example of a computer-readable storage medium. Computer memory is any memory which is directly accessible to a computational system. ‘Computer storage’ or ‘storage’ is a further example of a computer-readable storage medium. Computer storage is any non-volatile computer-readable storage medium. In some embodiments computer storage may also be computer memory or vice versa.

A ‘computational system’ as used herein encompasses an electronic component which is able to execute a program or machine executable instruction or computer executable code. References to the computational system comprising the example of “a computational system” should be interpreted as possibly containing more than one computational system or processing core. The computational system may for instance be a multi-core processor. A computational system may also refer to a collection of computational systems within a single computer system or distributed amongst multiple computer systems. The term computational system should also be interpreted to possibly refer to a collection or network of computing devices each comprising a processor or computational systems. The machine executable code or instructions may be executed by multiple computational systems or processors that may be within the same computing device or which may even be distributed across multiple computing devices.

Machine executable instructions or computer executable code may comprise instructions or a program which causes a processor or other computational system to perform an aspect of the present invention. Computer executable code for carrying out operations for aspects of the present invention may be written in any combination of one or more programming languages, including an object-oriented programming language such as Java, Smalltalk, C++ or the like and conventional procedural programming languages, such as the “C” programming language or similar programming languages and compiled into machine executable instructions. In some instances, the computer executable code may be in the form of a high-level language or in a pre-compiled form and be used in conjunction with an interpreter which generates the machine executable instructions on the fly. In other instances, the machine executable instructions or computer executable code may be in the form of programming for programmable logic gate arrays.

The computer executable code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described with reference to flowchart illustrations and/or block diagrams of methods, apparatus (systems) and computer program products according to embodiments of the invention. It is understood that each block or a portion of the blocks of the flowchart, illustrations, and/or block diagrams, can be implemented by computer program instructions in form of computer executable code when applicable. It is further under stood that, when not mutually exclusive, combinations of blocks in different flowcharts, illustrations, and/or block diagrams may be combined. These computer program instructions may be provided to a computational system of a general-purpose computer, special purpose computer, or other programmable data processing apparatus to produce a machine, such that the instructions, which execute via the computational system of the computer or other programmable data processing apparatus, create means for implementing the functions/acts specified in the flowchart and/or block diagram block or blocks.

These machine executable instructions or computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The machine executable instructions or computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

A ‘user interface’ as used herein is an interface which allows a user or operator to interact with a computer or computer system. A ‘user interface’ may also be referred to as a ‘human interface device.’ A user interface may provide information or data to the operator and/or receive information or data from the operator. A user interface may enable input from an operator to be received by the computer and may provide output to the user from the computer. In other words, the user interface may allow an operator to control or manipulate a computer and the interface may allow the computer to indicate the effects of the operator's control or manipulation. The display of data or information on a display or a graphical user interface is an example of providing information to an operator. The receiving of data through a keyboard, mouse, trackball, touchpad, pointing stick, graphics tablet, joystick, gamepad, webcam, headset, pedals, wired glove, remote control, and accelerometer are all examples of user interface components which enable the receiving of information or data from an operator.

A ‘hardware interface’ as used herein encompasses an interface which enables the computational system of a computer system to interact with and/or control an external computing device and/or apparatus. A hardware interface may allow a computational system to send control signals or instructions to an external computing device and/or apparatus. A hardware interface may also enable a computational system to exchange data with an external computing device and/or apparatus. Examples of a hardware interface include, but are not limited to: a universal serial bus, IEEE 1394 port, parallel port, IEEE 1284 port, serial port, RS-232 port, IEEE-488 port, Bluetooth connection, Wireless local area network connection, TCP/IP connection, Ethernet connection, control voltage interface, MIDI interface, analog input interface, and digital input interface.

A ‘display’ or ‘display device’ as used herein encompasses an output device or a user interface adapted for displaying images or data. A display may output visual, audio, and or tactile data. Examples of a display include, but are not limited to: a computer monitor, a television screen, a touch screen, tactile electronic display, Braille screen, Cathode ray tube (CRT), Storage tube, Bi-stable display, Electronic paper, Vector display, Flat panel display, Vacuum fluorescent display (VF), Light-emitting diode (LED) displays, Electroluminescent display (ELD), Plasma display panels (PDP), Liquid crystal display (LCD), Organic light-emitting diode displays (OLED), a projector, and Head-mounted display.

K-space data is defined herein as being the recorded measurements of radio frequency signals emitted by atomic spins using the antenna of a Magnetic resonance apparatus during a magnetic resonance imaging scan. Magnetic resonance data is an example of tomographic medical image data.

A Magnetic Resonance image or MR image is defined herein as being the reconstructed two-or three-dimensional visualization of anatomic data contained within the k-space data. This visualization can be performed using a computer.

### BRIEF DESCRIPTION OF THE DRAWINGS

In the following preferred embodiments of the invention will be described, by way of example only, and with reference to the drawings in which:

FIG. 1 illustrates an example of a medical system;

FIG. 2 shows a flowchart of a method of using the medical system of FIG. 1;

FIG. 3 illustrates an example of a medical system;

FIG. 4 shows a flowchart of a method of using the medical system of FIG. 3;

FIG. 5 shows a flowchart of a method of using the medical system of FIG. 1;

FIG. 6 illustrates an example of several different medical images before and after processing them by the trained machine learning model.

### DESCRIPTION OF EMBODIMENTS

Like numbered elements in these figures are either equivalent elements or perform the same function. Elements which have been discussed previously will not necessarily be discussed in later figures if the function is equivalent.

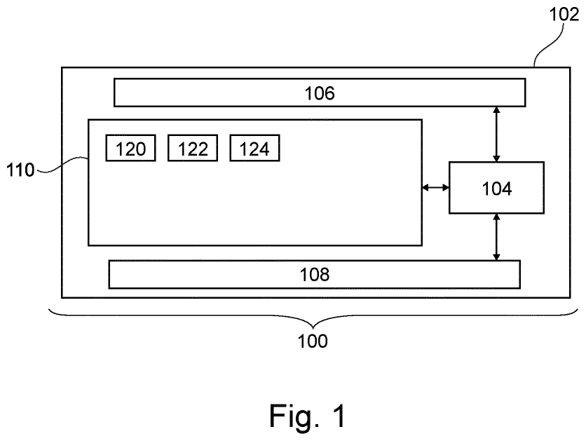

FIG. 1 illustrates an example of a medical system 100. In this example the medical system 100 comprises a computer 102 with a computational system 104. The computer 102 is intended to represent one or more computers or computer systems. The computational system 104 is intended to represent one or more computational systems or computational cores. The computer 102 is further shown as containing an optional hardware interface 106 which is connected to the computational system 104. If other components of the medical system 100 are present or included such as a magnetic resonance imaging system, then the hardware interface 106 could be used to exchange data and commands with these other components. The medical system 100 is further shown as comprising an optional user interface 108 which may provide various means for relaying data and receiving data and commands from an operator.

The medical system 100 is further shown as comprising a memory 110 that is connected to the computational system 104. The memory 110 is intended to represent various types of memory which may be accessible to the computational system 104. The memory 110 is shown as containing machine-executable instructions 120. The machine-executable instructions 120 are instructions which enable the computational system 104 to perform various control, data processing, and image processing tasks. The memory 110 is further shown as containing a machine learning model 122 and videos 124 obtained from one or more web sources.

The machine learning model 122 may be configured to receive as input a 3D medical image and to clean the 3D medical image. The cleaning may, for example, comprise denoising, anti-ringing or super resolution.

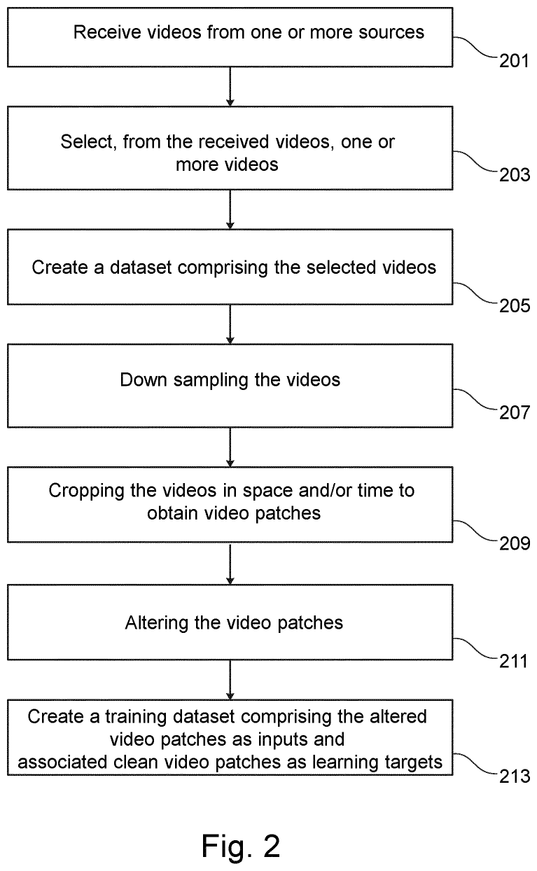

FIG. 2 is a flowchart of a method for creating training data for a machine learning model in accordance with an example of the present subject matter. The machine learning model is configured for obtaining an image of enhanced quality from an acquired 3D medical image. The method may for example be performed by the medical system of FIG. 1. Videos may be received in step 201 from one or more sources such as web sources. Videos having target values of image parameters may be selected in step 203 from the received videos. A dataset comprising the selected videos may be created in step 205. This may be referred to as initial dataset. The videos of the initial dataset may be down-sampled in step 207 by a predefined reduction factor. The down sampling may, for example, be performed along the spatial dimensions of the videos or along the time dimension of the videos. The down sampled videos of the initial dataset may be cropped in step 209 in space and/or time to obtain video patches, herein referred to as clean video patches. The clean video patches may be altered in step 211 for reducing a quality of the video patches. A training dataset may be created in step 213. The training dataset comprises the altered video patches as inputs and associated clean video patches as learning targets. Step 213 may, for example, further comprise storing the training dataset e.g., in the medical system 100 or in an accessible remote system.

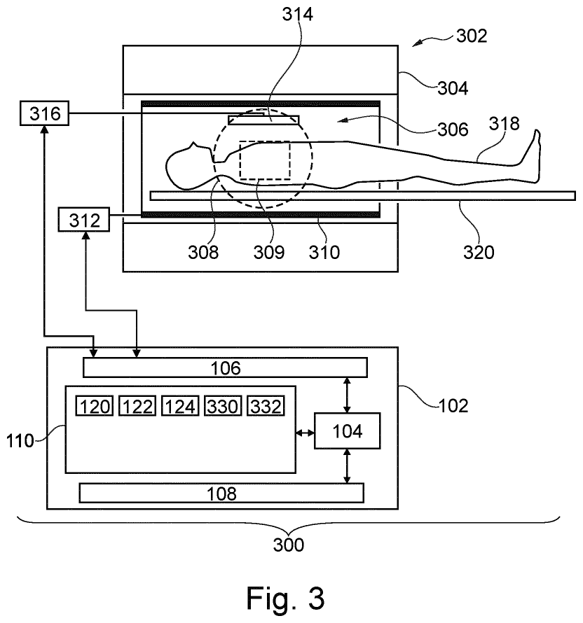

FIG. 3 illustrates a further example of the medical system 300. The medical system in FIG. 3 is similar to that as in FIG. 1 except that it additionally comprises a magnetic resonance imaging system 302 that is controlled by the computational system 104. The example illustrated in FIG. 3 is a magnetic resonance imaging system 302.

The magnetic resonance imaging system 302 comprises a magnet 304. The magnet 304 is a superconducting cylindrical type magnet with a bore 306 through it. The use of different types of magnets is also possible; for instance it is also possible to use both a split cylindrical magnet and a so called open magnet. A split cylindrical magnet is similar to a standard cylindrical magnet, except that the cryostat has been split into two sections to allow access to the iso-plane of the magnet, such magnets may for instance be used in conjunction with charged particle beam therapy. An open magnet has two magnet sections, one above the other with a space in-between that is large enough to receive a subject: the arrangement of the two sections area similar to that of a Helmholtz coil. Open magnets are popular, because the subject is less confined. Inside the cryostat of the cylindrical magnet there is a collection of superconducting coils.

Within the bore 306 of the cylindrical magnet 304 there is an imaging zone 308 where the magnetic field is strong and uniform enough to perform magnetic resonance imaging. A field of view 309 is shown within the imaging zone 308. The k-space data that is acquired typically acquired for the field of view 309. The region of interest could be identical with the field of view 309 or it could be a sub volume of the field of view 309. A subject 318 is shown as being supported by a subject support 320 such that at least a portion of the subject 318 is within the imaging zone 308 and the field of view 309.

Within the bore 306 of the magnet there is also a set of magnetic field gradient coils 310 which is used for acquisition of preliminary k-space data to spatially encode magnetic spins within the imaging zone 308 of the magnet 304. The magnetic field gradient coils 310 connected to a magnetic field gradient coil power supply 312. The magnetic field gradient coils 310 are intended to be representative. Typically, magnetic field gradient coils 310 contain three separate sets of coils for spatially encoding in three orthogonal spatial directions. A magnetic field gradient power supply supplies current to the magnetic field gradient coils. The current supplied to the magnetic field gradient coils 310 is controlled as a function of time and may be ramped or pulsed.

Adjacent to the imaging zone 308 is a radio-frequency coil 314 for manipulating the orientations of magnetic spins within the imaging zone 308 and for receiving radio transmissions from spins also within the imaging zone 308. The radio frequency antenna may contain multiple coil elements. The radio frequency antenna may also be referred to as a channel or antenna. The radio-frequency coil 314 is connected to a radio frequency transceiver 316. The radio-frequency coil 314 and radio frequency transceiver 316 may be replaced by separate transmit and receive coils and a separate transmitter and receiver. It is understood that the radio-frequency coil 314 and the radio frequency transceiver 316 are representative. The radio-frequency coil 314 is intended to also represent a dedicated transmit antenna and a dedicated receive antenna. Likewise the transceiver 316 may also represent a separate transmitter and receivers. The radio-frequency coil 314 may also have multiple receive/transmit elements and the radio frequency transceiver 316 may have multiple receive/transmit channels.

The transceiver 316 and the gradient controller 312 are shown as being connected to the hardware interface 106 of the computer system 102.

The memory 110 is further shown as containing pulse sequence commands 330. The pulse sequence commands 330 are commands or data which can be converted into commands which enable the computational system 104 to control the magnetic resonance imaging system to acquire k-space data 332. The memory 110 shows the k-space data 332 that has been acquired by controlling the magnetic resonance imaging system with the pulse sequence commands 330.

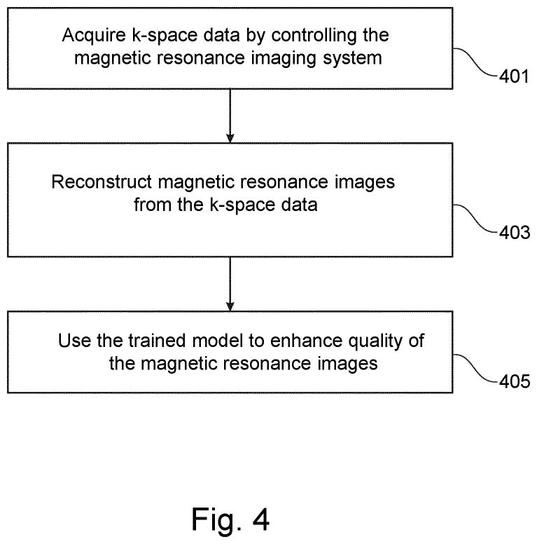

FIG. 4 is a flowchart of a method for acquiring MR image data in accordance with an example of the present subject matter. The method may for example be performed by the system of FIG. 3. For example, a machine learning model may be trained using the training dataset obtained by the method FIG. 2. The machine learning model may be configured to clean 3D MR images.

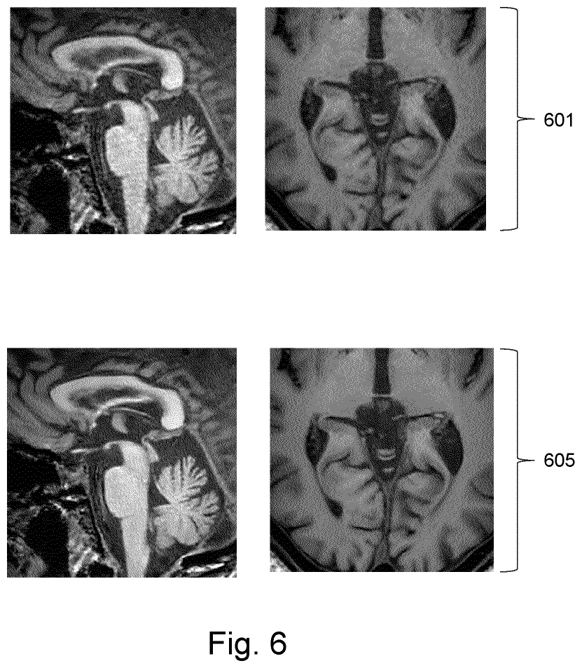

k-space data may be acquired in step 401 by controlling the magnetic resonance imaging system. 3D magnetic resonance images may be reconstructed in step 403 from the k-space data. The trained machine learning model may be used in step 405 to enhance quality of the 3D magnetic resonance images. FIG. 6 depicts an example of reconstructed images 601 before being denoised and corresponding images 605 which are denoised using the trained machine learning model. That is, the denoised images are obtained by inference of the trained machine learning model. Natural images (e.g., of the received videos) may not have complex part in it, while MR scans do. To overcome this for the inference, real and imaginary parts of the MR scan may be treated as individual images and both outputs are summed up to get the result by the end.

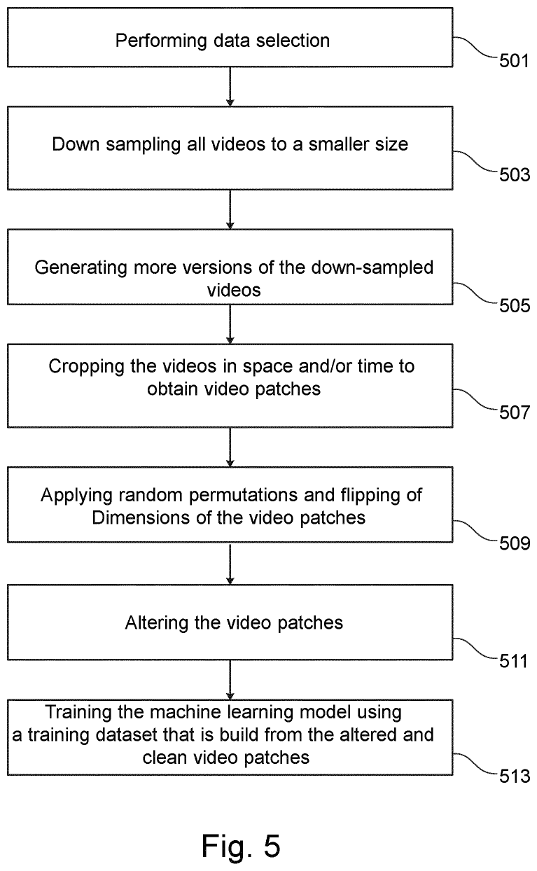

FIG. 5 is a flowchart of a method for creating training data for a machine learning model in accordance with an example of the present subject matter.

Natural high-quality videos (initial videos) may be obtained from web sources and used for training a machine learning model. The machine learning model may, for example, be integrated in an IQ Boost 3D system. The machine learning model may, for example be a Convolutional Neural Network that may be trained for the denoising, anti-ringing and super resolution tasks. A data selection may be performed in step 501. For example, videos may be selected from the initial videos. A video may be selected such that it is of high resolution, it should not contain artifacts from compression or interpolation, it should contain limited amount of noise. And, the gradients for each direction in the selected video should not be zero, this means that videos should contain some sort of motion and do not comprise of static images over time. Data may also be selected based on its license policy, so it could be used for product development purposes.

A Pre-Processing of the selected videos may be performed. In particular, the following steps may be an embodiment of the pre-processing; however, variations in pre-processing may occur. Videos were treated as three dimensional volumes where the third dimension was time. As an example, the pre-processing steps may be used for IQ Boost 3D, but the pre-processing is not restricted to these specific steps. All videos may be down-sampled in step 503 to a smaller matrix size that leads to structure appearance that is more similar to MRI scans. The temporal dimension is left as-is. To preserve good quality, a high-quality interpolator such as Lanczos may be used. As additional data augmentation, more versions of the videos may be generated in step 505 by resampling to different matrix sizes.

A random patch of small size from each dimension is cropped in step 507 from the normalized video. This step may allow to deal with huge volume sizes. In some embodiments, a small but fixed patch size may be used. Random permutations and flipping of dimensions are applied in step 509 to reduce the influence of the temporal dimension. This may be critical as the temporal dimension is very different from spatial dimensions. By randomly permuting and flipping, one may prevent the AI models from overfitting to properties of the temporal dimension. E.g., these steps make the video more similar to MR data properties.

Preparation of the input data of the machine learning model, may depend on the model purpose as it includes the alteration step. The video patches may be altered in step 511 in accordance with the purpose of the machine leavening model. A training dataset may be created from the video patches and the machine learning model may be trained in step 513 using the training dataset.

The machine learning model may be referred to as 3D AI model. The 3D AI model can be a fully convolutional neural network with 3D kernels or a ResNet, DenseNet, EfficientNet, or Vision Transformer with adjusted kernels for 3D processing. As an example of ML model, the 3D AI denoiser may be trained by adding random Gaussian noise to the clean pre-processed videos which are then used as input to the model. The learning target is the clean pre-processed clean video without noise. Training is performed with standard gradient descent methods. Another example is the 3D AI anti-ringing and super-resolution model. The model may be trained by applying a k-space crop to the clean pre-processed video, resulting in a low-resolution video with ringing artefacts that serves as an input to the model. The learning target is the clean, pre-processed video.

1. A medical system for creating training data for a machine learning model, the machine learning model being configured for improving quality of an acquired 3D medical image, the medical system comprising: a non-transitory memory configured to store machine executable instructions; and a computational system, wherein execution of the machine executable instructions causes the computational system to:
receive videos from one or more sources;
select, from the received videos, videos having desired values of one or more image parameters;
create a dataset comprising the selected videos;
pre-process the videos of the dataset, the pre-processing comprising
down sampling the videos by a predefined reduction factor;
cropping the down sampled videos in space and/or time to obtain video patches, herein referred to as clean video patches;
altering the clean video patches for reducing a quality of the clean video patches;
create a training dataset comprising the altered video patches as inputs and associated clean video patches as learning targets.

2. The medical system of claim 1, wherein execution of the machine executable instructions causes the computational system to permute the dimensions in selected video patches of the clean video patches before the alteration of the clean video patches.

3. The medical system of claim 1, wherein execution of the machine executable instructions causes the computational system to flip selected video patches of the clean video patches before the alteration of the clean video patches.

4. The medical system of claim 1, wherein the desired values of the image parameters comprise at least any of the following: a resolution higher than a minimum resolution, missing artifacts from compression or interpolation, an amount of noise smaller than a maximum amount, and the variation of the gradients for each direction in the video is larger than a minimum amount.

5. The medical system of claim 1, wherein execution of the machine executable instructions causes the computational system to alter the video patch by adding a random Gaussian noise to the video patch; and/or lowering a resolution of the video patch.

6. The medical system of claim 1, wherein execution of the machine executable instructions causes the computational system to randomly perform the crop such that each video patch has a size that is smaller than a maximum size.

7. The medical system of claim 1, the received videos being videos of non-medical fields.

8. The medical system of claim 1, wherein execution of the machine executable instructions causes the computational system to apply an interpolator for performing the down-sampling of the videos.

9. The medical system of claim 1, wherein execution of the machine executable instructions causes the computational system to: augment the dataset by resampling each video of the dataset to obtain different versions of the video, each version of the video having a different size; and add the obtained videos to the dataset before the pre-processing.

10. The medical system of claim 1, the reduction factor being defined such that the resolution obtained after down sampling the videos is similar or equal to a resolution of the 3D medical images of the machine learning model.

11. The medical system of claim 1, wherein execution of the machine executable instructions causes the computational system to train the machine learning model using the training dataset.

12. The medical system of claim 11, wherein the medical system further comprises a magnetic resonance imaging system, wherein the memory further contains pulse sequence commands configured to control the magnetic resonance imaging system to acquire k-space data according to the magnetic resonance imaging protocol, wherein execution of the machine executable instructions further causes the computational system to: acquire the k-space data by controlling the magnetic resonance imaging system; reconstruct 3D magnetic resonance images from the k-space data; and use the trained model to enhance quality of the 3D magnetic resonance images.

13. A computer program comprising machine executable instructions stored on a non-transitory computer readable medium, wherein execution of the machine executable instructions causes a computational system to:
receive videos from one or more sources;
select, from the received videos, videos having desired values of one or more image parameters;
create a dataset comprising the selected videos;
pre-process the videos of the dataset, the pre-processing comprising
down sampling the videos by a predefined reduction factor;
cropping the down sampled videos in space and/or time to obtain video patches, herein referred to as clean video patches;
altering the clean video patches for reducing a quality of the clean video patches;
create a training dataset comprising the altered video patches as inputs and associated clean video patches as learning targets.

14. A method for creating training data for a machine learning model, the machine learning model being configured for improving quality of an acquired 3D medical image, the method comprising:
receiving videos from one or more sources;
selecting, from the received videos, videos having desired values of one or more image parameters;
creating a dataset comprising the selected videos;
pre-processing the videos of the dataset, the pre-processing comprising
down sampling the videos by a predefined reduction factor;
cropping the down sampled videos in space and/or time to obtain video patches, herein referred to as clean video patches;
altering the clean video patches for reducing a quality of the clean video patches;
creating a training dataset comprising the altered video patches as inputs and associated clean video patches as learning targets.

15. A computer implemented data structure configured to train a machine learning model for cleaning 3D medical images, the data structure comprising altered video patches and associated clean video patches, wherein the altered video patch has an alteration obtained in accordance with a cleaning purpose of the machine learning model.

16. The data structure of claim 15, the altered video patches comprise noisy video patches or low-resolution video patches with ringing artefacts.
